(19) United States
(12) Patent Application Publication
(10) Pub. No.: US 2021/0241147 A1
(43) Pub. Date: Aug. 5, 2021

(54) METHOD AND DEVICE FOR PREDICTING PAIR OF SIMILAR QUESTIONS AND ELECTRONIC EQUIPMENT

(71) Applicant: Beijing More Health Technology Group Co. Ltd., Beijing (CN)

(72) Inventors: Dejie CHANG, Beijing (CN); Bangchang LIU, Beijing (CN); Shufeng GU, Beijing (CN); Hongwen ZHAO, Beijing (CN); Xiaobin LUO, Beijing (CN); Yikun ZHANG, Beijing (CN); Yunzhao WU, Beijing (CN); Chaozhen LIU, Beijing (CN); Hai WANG, Beijing (CN); Hangfei ZHANG, Beijing (CN); Ke JI, Beijing (CN)

(73) Assignee: Beijing More Health Technology Group Co. Ltd., Beijing (CN)

(21) Appl. No.: 17/238,169

(22) Filed: Apr. 22, 2021

Related U.S. Application Data
(63) Continuation of application No. PCT/CN2021/083022, filed on Mar. 25, 2021.

(30) Foreign Application Priority Data
Nov. 2, 2020 (CN) 202011200385.8

(51) Int. Cl. G06N 5/04; G06K 9/62

(52) U.S. Cl. CPC G06N 5/04; G06K 9/6215; G06K 9/6231; G06K 9/6256

(57) ABSTRACT

An embodiment of the present application provides a method and a device for predicting a pair of similar questions and an electronic equipment, wherein a pair of similar questions to be predicted are input into multiple different prediction models, and a prediction result output by each of the prediction models is obtained; a random disturbance parameter is added into an embedding layer of at least one of the prediction models; and voting operation is performed on multiple prediction results to obtain a final prediction result of the pair of similar questions to be predicted. According to the present application, the a random disturbance parameter is added into the embedding layer of the prediction model, so that over-fitting caused by over-learning of sample knowledge by the prediction model can be effectively prevented, and the prediction accuracy can be effectively improved by predicting the pair of similar questions utilizing the prediction model.

### CROSS-REFERENCE TO RELATED APPLICATION

This application is a continuation of PCT application NO. PCT/CN2021/083022 filed on Mar. 25, 2021, which claims the priority benefit of China application No. 202011200385.8 filed on Nov. 2, 2020. The entirety of the above-mentioned patent applications is hereby incorporated by reference herein and made a part of this specification.

### TECHNICAL FIELD

The present application relates to the technical field of neural network models, in particular to a method and a device for predicting a pair of similar questions and an electronic equipment.

### BACKGROUND ART

It is a valuable thing to use neural network classification models to classify common questions and answers of patients by similarity, such as identifying similar questions of patients, helping to understand patients' real appeal, helping to quickly match accurate answers and improving patients' sense of acquisition. Conclusion of doctors' similar answers can help analyzing the standardization of answers and avoid misdiagnosis.

At present, fixed disturbance parameters are often added to the existing neural network classification models to prevent over-fitting, but it is easy to learn sample knowledge in the process of model training in this way, which is disadvantageous for preventing over-fitting.

### SUMMARY

Accordingly, some embodiments of the present application provide a method and a device for predicting a pair of similar questions and an electronic equipment in order to alleviate the technical questions.

In a first aspect, an embodiment of the present application provides a method for predicting a pair of similar questions, wherein the method comprises: inputting a pair of similar questions to be predicted into multiple different prediction models to obtain a prediction result output by each of the prediction models; adding a random disturbance parameter into an embedding layer of at least one of the prediction models; and performing voting operation on multiple prediction results to obtain a final prediction result of the pair of similar questions to be predicted.

In combination with the first aspect, an embodiment of the present application provides a first possible implementation of the first aspect, wherein each of the prediction models comprises multiple prediction sub-models, wherein each of the prediction sub-models is obtained by training the prediction model from a specific training sample set of the pair of similar questions and a training sample set of the pair of similar questions determined by an allocation function; a step of obtaining a prediction result output by each of the prediction models, comprising: inputting the pair of similar questions to be predicted into multiple prediction sub-models included in each of the prediction models to obtain a prediction sub-result output by each of the prediction sub-models; performing voting operation on multiple prediction sub-results to obtain the prediction results.

In combination with the first possible implementation of the first aspect, an embodiment of the present application provides a second possible implementation of the first aspect, wherein the prediction sub-model is trained in the following manner, which comprises: obtaining an original training sample set of the pair of similar questions; performing training sample extension processing on the original training sample set of the pair of similar questions by utilizing a similarity transmission principle to obtain an extended training sample set of the pair of similar questions; determining the training sample set of the pair of similar questions from the extended training sample set of the pair of similar questions based on the allocation function; training the prediction model by utilizing the training sample set of the pair of similar questions and the specific training sample set of the pair of similar questions to obtain the prediction sub-model.

In combination with the second possible implementation of the first aspect, an embodiment of the present application provides a third possible implementation of the first aspect, wherein, after an extended training sample set of the pair of similar questions is obtained, the method further comprises: sequentially labeling each pair of training samples of the pair of similar questions in the extended training sample set of the pair of similar questions; a step of determining the training sample set of the pair of similar questions from the extended training sample set of the pair of similar questions based on the allocation function, comprising: determining a first label from the extended training sample set of the pair of similar questions by utilizing a first function of the allocation function: determining a second label from the extended training sample set of the pair of similar questions based on the first label by utilizing a second function of the allocation function: and selecting an extended training sample set of the pair of similar questions between the first label and the second label as the training sample set of the pair of similar questions.

In combination with the third possible implementation of the first aspect, an embodiment of the present application provides a fourth possible implementation of the first aspect, wherein the first function is: i=AllNumber*radom (0,1)+offset; wherein i represents the first label, i<AllNumber, AllNumber indicates a length of the extended training sample set of the pair of similar questions, offset represents an offset, offset <AllNumber, and the offset is a positive integer.

In combination with the third possible implementation of the first aspect, an embodiment of the present application provides a fifth possible implementation of the first aspect, wherein the second function is: j=i+A %*AllNumber; wherein j represents the second label, i≤j<AllNumber, A is a positive integer, 0≤A≤100, i represents the first label, and AllNumber indicates a length of the extended training sample set of the pair of similar questions.

In combination with the second possible implementation of the first aspect, an embodiment of the present application provides a sixth possible implementation of the first aspect, wherein the similarity between each pair of specific training samples of the pair of similar questions in the specific training sample set of the pair of similar questions and the training sample set of the pair of similar questions is greater than a preset similarity; a step of training the prediction model by utilizing the training sample set of the pair of similar questions and the specific training sample set of the pair of similar questions to obtain the prediction sub-model, comprising: training a first preset network layer number parameter of the prediction model based on the training sample set of the pair of similar questions, and obtaining a prediction preliminary model of the prediction model when a loss function of the prediction model converges; and training a second preset network layer number parameter of the prediction preliminary model based on the specific training sample set of the pair of similar questions, and obtaining the prediction sub-model when the loss function of the prediction preliminary model converges.

In combination with the first aspect, an embodiment of the present application provides a seventh possible implementation of the first aspect, wherein the random disturbance parameter is generated utilizing the following formula: delta=1/1+exp(−a); wherein delta represents the random disturbance parameter and a represents a parameter factor, −5≤a≤5.

In a second aspect, an embodiment of the present application also provides a device for predicting a pair of similar questions, wherein the device comprises: an input module used for inputting a pair of similar questions to be predicted into multiple different prediction models to obtain a prediction result output by each of the prediction models; and adding a random disturbance parameter into an embedding layer of at least one of the prediction models; and an operation module used for performing voting operation on multiple prediction results to obtain a final prediction result of the pair of similar questions to be predicted;

In a third aspect, an embodiment of the present application also provides an electronic equipment comprising a processor and a memory, the memory storing computer-executable instructions executable by the processor, the processor executing the computer-executable instructions to implement the method described above.

The embodiment of the present application brings the following beneficial effects.

An embodiment of the present application provides a method and a device for predicting a pair of similar questions and an electronic equipment, wherein a pair of similar questions to be predicted are input into multiple different prediction models, and a prediction result output by each of the prediction models is obtained; a random disturbance parameter is added into an embedding layer of at least one of the prediction models; and voting operation is performed on multiple prediction results to obtain a final prediction result of the pair of similar questions to be predicted. According to the present application, the a random disturbance parameter is added into the embedding layer of the prediction model, so that over-fitting caused by over-learning of sample knowledge by the prediction model can be effectively prevented, and the prediction accuracy can be effectively improved by predicting the pair of similar questions utilizing the prediction model.

Additional features and advantages of the present application will be set forth in the description which follows, and in part will be obvious from the description, or may be learned by practice of the present application. The objectives and other advantages of the present application may be realized and attained by the structure particularly pointed out in the written description and drawings.

The objects, features and advantages of the present application will become more apparent from the following detailed description, taken in combination with the accompanying drawings, in which preferred embodiments are set forth.

### BRIEF DESCRIPTION OF THE DRAWINGS

In order that the detailed implementations of the present application or the technical solutions in the prior art may be more clearly described, reference will now be made to the accompanying drawings which is used in description of the detailed implementations or the prior art. It is obvious that the drawings in the following description are some embodiments of the present application, and that those skilled in the art can obtain other drawings from these drawings accordingly without involving any inventive effort.

FIG. 1 is a flow diagram of a method for predicting a pair of similar questions provided by an embodiment of the present application;

FIG. 2 is a schematic diagram of a training sample extension provided by an embodiment of the present application;

FIG. 3 is a flow diagram of another similar method for predicting a pair of similar questions provided by an embodiment of the present application;

FIG. 4 is a schematic diagram of a structure of a device for predicting a pair of similar questions provided by an embodiment of the present application; and

FIG. 5 is a schematic diagram of an electronic equipment provided by an embodiment of the present application.

### DETAILED DESCRIPTION

In order that the purposes, technical solutions, and advantages of the embodiments of the present application will become more apparent, in the following context, taken in conjunction with the accompanying drawings, more clear and complete description will be made to the technical solutions of the present application. It is to be understood that the described embodiments are only a few, but not all, embodiments of the disclosure. Based on the embodiments of the present application, all other embodiments obtained by a person skilled in the art without involving any inventive effort are within the scope of the present application.

At present, fixed disturbance parameters are often added to the existing neural network classification models to prevent over-fitting. However, in this way, it is easy to learn sample knowledge in the model training process, which is disadvantageous for preventing over-fitting. On such basis, in the method and device for predicting a pair of similar questions and the electronic equipment provided by the embodiment of the present application, the random disturbance parameter is added into the embedding layer of the prediction model, so that over-fitting caused by over-learning of sample knowledge of the prediction model can be effectively prevented, and the prediction accuracy can be effectively improved by utilizing the prediction model to predict the pair of similar questions.

To facilitate an understanding of the present embodiment, a method for predicting similar questions as disclosed in the embodiment of the present application is first described in detail.

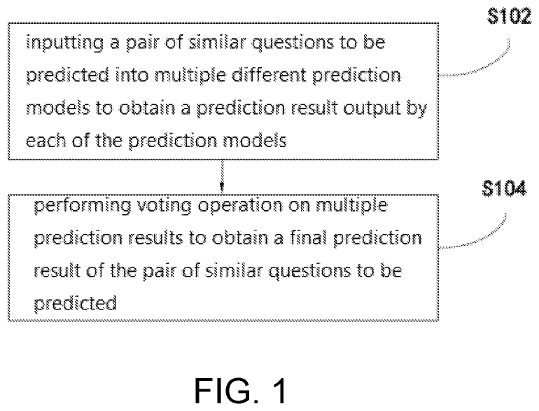

With reference to the flow diagram of the method for predicting similar questions shown in FIG. 1, the method specifically includes the following steps:

Step S102, inputting a pair of similar questions to be predicted into multiple different prediction models to obtain a prediction result output by each of the prediction models; adding a random disturbance parameter into an embedding layer of at least one of the prediction models;

a pair of similar questions refer to a group of a pair of similar questions composed of two relatively similar questions, such as “how does hemoptysis happen after strenuous movement” and “why hemoptysis occurs after strenuous movement”, which constitute a group of a pair of similar questions. “how does hemoptysis happen after strenuous movement” and “what is to be done with hemoptysis after strenuous movement” constitute a group of a pair of similar questions.

In general, different prediction models refer to different types of prediction models, and three text classification models with different prediction types, namely a roberta wwm large model, a roberta pair large model and an ernie model, as commonly seen, can be selected as a prediction model to predict a pair of similar questions to be predicted so as to respectively obtain prediction results output by the three prediction models. The determination of the prediction model may be chosen according to practical needs and is not limited here.

According to the prediction result, it can be determined that the prediction model predicts whether the pair of similar questions to be predicted belong to a group of questions with the same meaning or belong to a group of questions with different meanings. If the obtained prediction result is 0, the meanings are the same, and if the obtained prediction result is 1, the meanings are different. The meaning of the prediction result can be set as required and is not limited herein.

In the embodiment, the random disturbance parameter can be added to the embedding layer of at least one of the three prediction models, the over-fitting caused by over-learning of training sample knowledge of the prediction model in the model training process can be prevented, and the prediction capability of the prediction model can be effectively improved.

Specifically, the random disturbance parameter is generated utilizing the following formula:

$$delta = \frac{1}{1 + exp ( - a )} ;$$

wherein delta represents the random disturbance parameter and a represents a parameter factor, −5≤a≤5.

S104, performing voting operation on multiple prediction results to obtain a final prediction result of the pair of similar questions to be predicted.

In the present embodiment, the voting operation may adopt an absolute majority voting method (more than half votes), a relative majority voting method (most votes) or a weighted voting method, and the specific voting method may be determined according to actual needs and is not limited herein.

In the embodiment, voting operation is performed on output prediction results of the three prediction models by utilizing a relative majority voting method to obtain a final prediction result of the pair of similar questions to be predicted. For example, a prediction result obtained by inputting a pair of similar questions to be predicted into the roberta wwm large model is 0, a prediction result obtained by inputting a pair of similar questions to be predicted into the roberta pair large model is 0, and a prediction result obtained by inputting a pair of similar questions to be predicted into the ernie model is 1. A final prediction result obtained based on the relative majority voting method is 0, which means that the pair of similar questions to be predicted are in a group of question pairs with the same meaning.

An embodiment of the present application provides a method for predicting a pair of similar questions, wherein a pair of similar questions to be predicted are input into multiple different prediction models, and a prediction result output by each of the prediction models is obtained; a random disturbance parameter is added into an embedding layer of at least one of the prediction models; and voting operation is performed on multiple prediction results to obtain a final prediction result of the pair of similar questions to be predicted. According to the present application, the a random disturbance parameter is added into the embedding layer of the prediction model, so that over-fitting caused by over-learning of sample knowledge by the prediction model can be effectively prevented, and the prediction accuracy can be effectively improved by predicting the pair of similar questions utilizing the prediction model.

In general, each of the prediction models comprises multiple prediction sub-models, wherein each of the prediction sub-models is obtained by training the prediction model a training sample set of the pair of similar questions determined by an allocation function. Specifically, the training process of the prediction sub-model can be realized by steps A1-A4:

Step A1, obtaining an original training sample set of the pair of similar questions;

The original training sample set of the pair of similar questions can be a denoised and cleaned original training sample set of the pair of similar questions which is obtained from a network or other storage equipment in advance. In actual use, the original training sample set of the pair of similar questions can be subjected to characteristic exploration and characteristic distribution exploration, the main means to be performed are exploration, category distribution, sentence length distribution exploration and the like, data analysis can be performed according to the explored characteristics, and research on subsequent training of the prediction model is facilitated.

Step A2, performing training sample extension processing on the original training sample set of the pair of similar questions by utilizing a similarity transmission principle to obtain an extended training sample set of the pair of similar questions;

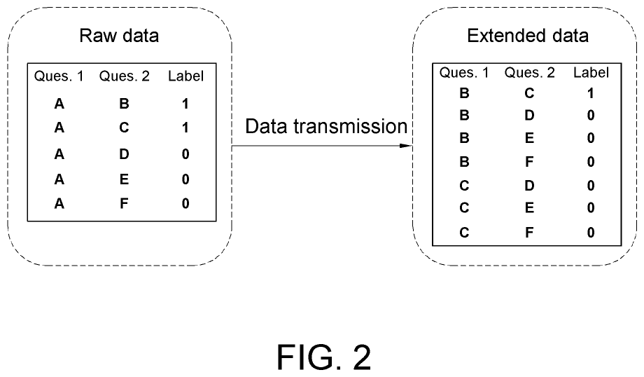

The original training sample set of the pair of similar questions are all labeled training samples to be used for training the prediction model, and for the convenience of understanding, FIG. 2 shows a schematic diagram of training sample extension. As shown in the left most block of FIG. 2, for the collected original training sample set of the pair of similar questions, query 1 (question 1), query 2 (question 1) and label (label) form a group of training samples. For example, A and B in the first row correspond to label 1, indicating that question A and question B are a group of question pairs with different meanings. A and C in the second row correspond to label 1, indicating that question A and question C are a group of question pairs with different meanings. A and D in the third row, A and E in the fourth row, and A and F in the fifth row correspond to label 0, which means that A and D are a group of question pairs with the same meaning, A and E are a group of question pairs with the same meaning, and A and F are a group of question pairs with the same meaning.

The content shown in the right-hand block of FIG. 2 is extended data with training sample extension processing performed on the original training sample set of the pair of similar questions in the left-hand block by utilizing a similarity transmission principle. Specifically, it can be seen from a first row of training samples and a second row of training samples in the original training sample set of the pair of similar questions that A and B are a group of question pairs with different meanings, and A and C are a group of question pairs with different meanings as well. It can be inferred that B and C are a group of question pairs with different meanings. According to the first row of training samples and the third row of training samples in the original training sample set of the pair of similar questions, since A and D are a group of question pairs with the same meaning, B and D are a group of question pairs with the same meaning. In the same way, the extended data after the original training sample set of the pair of similar questions is derived and transmitted according to the similarity transmission principle in the right-hand block of FIG. 2 can be obtained, and the derivation of the remaining extended data in the right-hand block of FIG. 2 is not described in detail.

In order to ensure that there is little difference between the 0/1 label distribution ratio of the extended training sample set of the pair of similar questions and the training sample set of the pair of similar questions, the 0/1 label distribution ratio of the extended data and the original training sample set of the pair of similar questions which can be selected in the right-hand block of FIG. 2 is close to the 0/1 label distribution ratio of the original training sample set of the pair of similar questions. Since the 0/1 label distribution ratio of the original training sample set of the pair of similar questions is 2:3, extended data with a group of labels of 1 and a group of labels of 0 in the right-hand block of FIG. 2 can be selected and added to the original training sample set of the pair of similar questions to form an extended training sample set of the pair of similar questions, so that the 0/1 label distribution ratio of the extended training sample set of the pair of similar questions to guarantee that the 0/1 label distribution ratio (3:4) of the extended training sample set of the pair of similar questions is close to the 0/1 label distribution ratio of the original training sample set of the pair of similar questions. In particular, any one of the rows of the extended data from the first row of the extended data and the remaining six rows of extended data in the right-hand block of FIG. 2 can be selected and added to the original training sample set of the pair of similar questions to form an extended training sample set of the pair of similar questions for training the prediction sub-model.

Step A3, determining the training sample set of the similar pair problems from the extended training sample set of the similar pair problems based on the allocation function;

Generally, before determining the training sample set of the pair of similar questions, each pair of training samples of the pair of similar questions in the extended training sample set of the pair of similar questions need to be sequentially labeled. For example, there are 100 question pairs in the extended training sample set of the pair of similar questions, and the 100 question pairs are sequentially labeled as 0-100.

The process of step A3 can be implemented by steps B1-B3:

Step B1, determining a first label from the extended training sample set of the similar pair problems by utilizing a first function of the allocation function:

In particular, the first function is: i=AllNumber*radom (0,1)+offset where i represents the first label, i<AllNumber, AllNumber indicates a length of the extended training sample set of the similar pair problems, offset represents an offset, offset <AllNumber, and the offset is a positive integer.

Continuing with the example of a total of 100 question pairs in the extended training sample set of the pair of similar questions, the length of AllNumber is 100 and the offset is set to 10. If the random number of radom (0,1) is 0.1 when the first label is determined for the first time, the first label calculated by the first function is i=20. Here, the offset can be set according to actual needs, and is not limited thereto.

Step B2, determining a second label from the extended training sample set of the similar pair problems based on the first label by utilizing a second function of the allocation function:

the second function is: j=i+A %*AllNumber wherein j represents the second label, i≤j≤AllNumber, A is a positive integer, and 0≤A≤100.

If A is set to 20, then j=40 is known from the resulting i=20. Here, A may be set according to actual needs, and is not limited thereto.

Step B3, selecting an extended training sample set of the similar pair problems between the first label and the second label as the training sample set of the similar pair problems.

After the first label and the second label are obtained through the allocation function, label matching is performed on the extended training sample set of the pair of similar questions sequentially labeled respectively, and the training samples in the interval from the label of 20 to the label of 40 in the extended training sample set of the pair of similar questions serve as a primary training sample set of the pair of similar questions.

Due to the existence of radom (0,1) in the allocation function, the training sample set for the pair of similar questions determined each time is also random.

Step A4, training the prediction model by utilizing the training sample set of the similar pair problems and the specific training sample set of the similar pair problems to obtain the prediction sub-model.

The specific training sample set of the pair of similar questions is training samples which are specifically collected according to an actual prediction question pair so as to enhance the prediction capability of a prediction sub-model. For example, for a medical question pair prediction, a pre-training model which simply depends on the above mentioned three prediction models (the three prediction models are all bert models) per se may not be enough, so that in this time, on the basis of the bert, via on-line acquisition of medical corpus samples, a medical bert is trained for pre-training enhancement.

The determination process of the specific training sample set of the pair of similar questions is as follows: a) widely collecting question pairs on a website; b) comparing the similarity with the question pairs in the extended training sample set of the pair of similar questions, and comparing the similarity by utilizing a Manhattan distance method, an Euclidean distance method, a Chebyshev distance method and the like, without limitation; medical corpus samples with similarity greater than a preset similarity are left to form a specific training sample set of the pair of similar questions.

The process of training the prediction model by utilizing the training sample set of the pair of similar questions and the specific training sample set of the pair of similar questions to obtain the prediction sub-model is as follows: training a first preset network layer number parameter of the prediction model based on the training sample set of the pair of similar questions until a loss function of the prediction model converges to obtain a prediction preliminary model of the prediction model; and training a second preset network layer number parameter of the prediction preliminary model based on the specific training sample set of the similar pair problems, and obtaining the prediction sub-model when the loss function of the prediction preliminary model converges.

For example, the first five layers of network parameters of the prediction model are trained by utilizing the training sample set of the pair of similar questions to obtain a prediction preliminary model, and the representation layer parameters of the bert are finely adjusted and trained by utilizing the selected specific training sample set of the pair of similar questions to obtain the prediction sub-model.

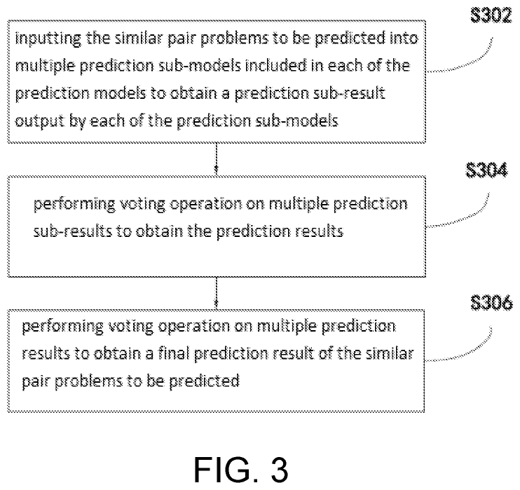

Based on the description of training of the prediction sub-model, the embodiment provides another method for predicting a pair of similar questions, which is realized on the basis of the embodiment. This embodiment focuses on the implementation of obtaining the prediction result output by each of the prediction models. As shown in the flow diagram of another method for predicting a pair of similar questions shown in FIG. 3, the method for predicting a pair of similar questions in the embodiment comprises the following steps:

Step S302, inputting the similar pair problems to be predicted into multiple prediction sub-models included in each of the prediction models to obtain a prediction sub-result output by each of the prediction sub-models;

The prediction model comprises multiple prediction sub-models which are obtained by training the prediction model (for example, the roberta wwm large model) by respectively utilizing multiple training sample sets of the pair of similar questions determined by the allocation function and the specific training sample set of the pair of similar questions, wherein the multiple prediction sub-models may have different internal parameters because the training sample set of the pair of similar questions may be different. Therefore, the prediction sub-results output by the multiple prediction sub-models may be different.

In the present embodiment, five training sample sets of the pair of similar questions determined by each of the prediction models utilizing the allocation function and a specific training sample set of the pair of similar questions are trained to obtain five prediction sub-models as an example. 15 prediction sub-models can be obtained via the three prediction models.

Step S304, performing voting operation on multiple prediction sub-results to obtain the prediction results;

Five prediction sub-models included in each of the prediction models are subjected to voting operation for once to obtain a prediction result corresponding to each of the prediction models, and five prediction sub-models of the roberta wwm large model are taken as examples for illustration, wherein the prediction sub-results obtained by the five prediction sub-models are 0, 0, 1, 0 and 0 respectively. When a relative majority voting method is adopted to perform voting operation, the prediction result of the roberta wwm large model is 0, and the prediction result of the roberta pair large model and the prediction result of the ernie model are the same as the prediction result obtained by the roberta wwm large model, and are not illustrated in detail here. The voting operation method can be selected according to actual requirements, and is not limited herein.

Step S306, performing voting operation on multiple prediction results to obtain a final prediction result of the similar pair problems to be predicted.

With regard to the roberta wwm large model, the roberta pair large model and the ernie model, after the prediction results are obtained by utilizing the prediction sub-results of multiple prediction sub-models respectively, voting operation is needed for once to obtain the final prediction result of the pair of similar questions to be predicted.

According to the method for predicting a pair of similar questions provided by the embodiment of the present application, firstly, the prediction result of each of the prediction models is obtained through a first voting operation of the prediction sub-results output by multiple prediction sub-models contained in each of the prediction models, and then the prediction results of the multiple prediction models are subjected to a secondary voting to obtain the final prediction result of the pair of similar questions to be predicted. According to the present application, after internal voting of the prediction model is finished, voting among the prediction models is performed to generate a final prediction result, the credibility of the model can be enhanced through secondary voting operation, and the prediction accuracy of the model can be improved.

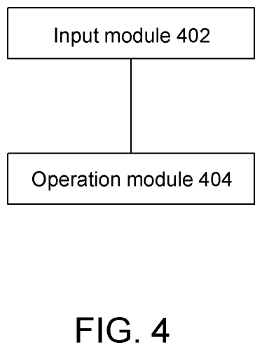

Corresponding to the method embodiment, an embodiment of the present application provides a device for predicting a pair of similar questions, and FIG. 4 shows a schematic diagram of a structure of the device for predicting a pair of similar questions.

The input module 402 is used for inputting similar pair problems to be predicted into multiple different prediction models to obtain a prediction result output by each of the prediction models; and adding a random disturbance parameter into an embedding layer of at least one of the prediction models;

The operation module 404 is used for performing voting operation on multiple prediction results to obtain a final prediction result of the similar pair problems to be predicted;

An embodiment of the present application provides a device for predicting a pair of similar questions, wherein similar pair problems to be predicted are input into multiple different prediction models, and a prediction result output by each of the prediction models is obtained; a random disturbance parameter is added into an embedding layer of at least one of the prediction models; and voting operation is performed on multiple prediction results to obtain a final prediction result of the similar pair problems to be predicted. According to the present application, the a random disturbance parameter is added into the embedding layer of the prediction model, so that over-fitting caused by over-learning of sample knowledge by the prediction model can be effectively prevented, and the prediction accuracy can be effectively improved by predicting the similar pair problems by utilizing the prediction model.

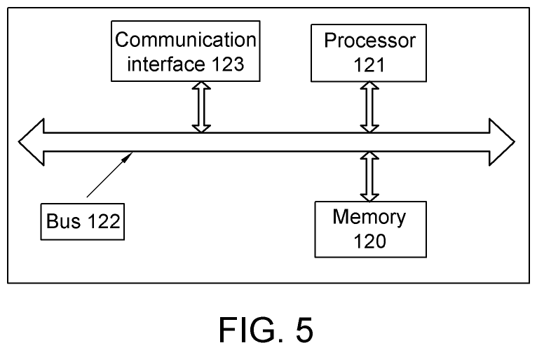

An embodiment of the present application also provides an electronic equipment, as shown in FIG. 5, which is a schematic diagram of a structure of the electronic equipment, wherein the electronic equipment includes a processor 121 and a memory 120, the memory 120 storing computer-executable instructions executable by the processor 121, the processor 121 executing the computer-executable instructions to implement the method for predicting a pair of similar questions described above.

In the embodiment shown in FIG. 5, the electronic equipment further comprises a bus 122 and a communication interface 123, wherein the processor 121, the communication interface 123 and the memory 120 are connected via the bus 122.

The memory 120 may include, among other things, high-speed random access memory (RAM), and may also include non-volatile memory, such as at least one disk memory. The communication connection between the system network element and at least one other network element is achieved via at least one communication interface 123, which may be wired or wireless, utilizing the Internet, a wide area network, a local network, a metropolitan area network, etc. Bus 122 may be an ISA (Industry Standard Architecture) bus, a PCI (Peripheral Component Interconnect) bus, an EISA (Extended Industry Standard Architecture) bus, or the like. The bus 122 may be divided into an address bus, a data bus, a control bus, etc. For ease of illustration, only one bi-directional arrow is shown in FIG. 5, but it is not limited to only one bus or one type of bus.

The processor 121 may be an integrated circuit chip having signal processing capabilities. In implementation, the steps of the method described above may be performed by integrated logic circuitry in hardware or instructions in software within the processor 121. The processor 121 may be a general-purpose processor including a central processing unit (CPU), a network processor (NP), etc; it may also be a digital signal processor (DSP), an application specific integrated circuit (ASIC), a field-programmable gate array (FPGA) or other programmable logic device, discrete gate or transistor logic device, or discrete hardware components. A general processor may be a microprocessor or the processor may be any conventional processor or the like. The steps of the method disclosed to combination with the embodiments of the present application may be embodied directly by being executed by a hardware decoding processor, or by a combination of hardware and software modules in the decoding processor. A software module may reside in random access memory, flash memory, read only memory, programmable read only memory, or electrically erasable programmable memory, registers, or other storage media as is well known in the art. The storage medium is located in the memory, and the processor 121 reads the information in the memory and, in combination with its hardware, performs the steps of the method for a pair of similar questions of the previous embodiment.

An embodiment of the present application also provides a computer-readable storage medium having stored thereon computer-executable instructions that, when invoked and executed by a processor, cause the processor to implement the method for a pair of similar questions. Implementations of the method may be found in the foregoing method embodiments and will not be described in detail herein.

A computer program product of the method and device for predicting a pair of similar questions and the electronic equipment provided by embodiments of the present application includes a computer readable storage medium storing program code including instructions operable to perform the methods described in the foregoing method embodiments, and specific implementations may be found in the method embodiments and will not be described in detail herein.

The relative steps, numerical expressions, and numerical values of the components and steps set forth in these examples do not limit the scope of the present application unless specifically stated otherwise.

The functions, if implemented in the form of software functional units and sold or used as stand-alone products, may be stored in a non-volatile computer-readable storage medium executable by a processor. Based on this understanding, the technical solution of the present application, either in essence or as part with contribution to the prior art or as part of the technical solution, may be embodied in the form of a software product stored in a storage medium comprising instructions for causing a computer device (which may be a personal computer, a server, or a network device, etc.) to perform all or part of the steps of the methods of the various embodiments of the present application. The above mentioned storage medium includes media such as: a U-disk, a removable hard disk, a read-only memory (ROM), a random access memory (RAM), a magnetic disk, or an optical disk which may store various program codes.

In the description of the present application, it should be noted that the orientation or positional relationships indicated by the terms “center”, “upper”, “lower”, “left”, “right”, “vertical”, “horizontal”, “inner”, “outer” and the like are based on the orientation or positional relationships shown in the drawings, merely to facilitate the description of the present application and to simplify the description. It is not intended to indicate or imply that the referenced device or element must have a particular orientation or be constructed and operated in a particular orientation, and thus should not be construed as limiting the present application. Furthermore, the terms “first”, “second”, and “third” are used for descriptive purposes only and are not to be construed as indicating or implying relative importance.

At last, the above-described embodiments are merely specific embodiments of the present application to illustrate the technical solutions of the present application and not to limit the scope of the present application which is not limited thereto. Although the present application has been described in detail with reference to the foregoing embodiments, those skilled in the art should understand that: any person skilled in the art, within the technical scope of the present application of the present application, can still make modifications to the technical solutions described in the foregoing embodiments or easily conceive changes, or make equivalent substitutions for some of the technical features thereof. Such modifications, variations or substitutions which do not make the essence of the corresponding technical solutions depart from the spirit and scope of the embodiments of the present application are intended to be within the scope of this application. Therefore, the scope of protection of this application should be determined with reference to the claims.

What is claimed is:

1. A method for predicting a pair of similar questions, comprising:
inputting a pair of similar questions to be predicted into multiple different prediction models to obtain a prediction result output by each of the prediction models;
adding a random disturbance parameter into an embedding layer of at least one of the prediction models; and
performing voting operation on multiple prediction results to obtain a final prediction result of the pair of similar questions to be predicted;
wherein each of the prediction models comprises a plurality of prediction sub-models, wherein each of the plurality of prediction sub-models is obtained by training the prediction model from a specific training sample set of the pair of similar questions and a training sample set of the pair of similar questions determined by an allocation function.

2. The method according to claim 1, wherein inputting the pair of similar questions to be predicted into multiple different prediction models to obtain the prediction result output by each of the prediction models comprises:
inputting the pair of similar questions to be predicted into multiple prediction sub-models included in each of the prediction models to obtain a prediction sub-result output by each of the prediction sub-models; and
performing voting operation on multiple prediction sub-results to obtain the prediction results.

3. The method according to claim 1, wherein the prediction sub-model is obtained by training the prediction model from the specific training sample set of the pair of similar questions and the training sample set of the pair of similar questions determined by the allocation function comprises:
obtaining an original training sample set of the pair of similar questions;
performing a training sample extension processing on the original training sample set of the pair of similar questions by utilizing a similarity transmission principle to obtain an extended training sample set of the pair of similar questions;
determining the training sample set of the pair of similar questions from the extended training sample set of the pair of similar questions based on the allocation function; and
training the prediction model by utilizing the training sample set of the pair of similar questions and the specific training sample set of the pair of similar questions to obtain the prediction sub-model.

4. The method according to claim 3, wherein after obtaining the extended training sample set of the pair of similar questions, the method further comprises:
sequentially labeling each pair of training samples of the pair of similar questions in the extended training sample set of the pair of similar questions.

5. The method according to claim 3, wherein determining the training sample set of the pair of similar questions from the extended training sample set of the pair of similar questions based on the allocation function comprises:
determining a first label from the extended training sample set of the pair of similar questions by utilizing a first function of the allocation function;
determining a second label from the extended training sample set of the pair of similar questions based on the first label by utilizing a second function of the allocation function; and
selecting an extended training sample set of the pair of similar questions between the first label and the second label as the training sample set of the pair of similar questions.

6. The method according to claim 5, wherein the first function is:
i=AllNumber*radom(0,1)+offset;
wherein i represents the first label, i<AllNumber, AllNumber indicates a length of the extended training sample set of the pair of similar questions, offset represents an offset, offset <AllNumber,
and the offset is a positive integer.

7. The method according to claim 6, wherein the second function is:
j=i+A%*AllNumber;
wherein j represents the second label, i≤j≤AllNumber, A is a positive integer, and 0≤A≤100.

8. The method according to claim 1, wherein the similarity between each pair of specific training samples of the pair of similar questions in the specific training sample set of the pair of similar questions and the training sample set of the pair of similar questions is greater than a preset similarity; and
the step of obtaining the prediction sub-model by training the prediction model by utilizing the training sample set of the pair of similar questions and the specific training sample set of the pair of similar questions comprises:
training a first preset network layer number parameter of the prediction model based on the training sample set of the pair of similar questions, and obtaining a prediction preliminary model of the prediction model when a loss function of the prediction model converges; and
training a second preset network layer number parameter of the prediction preliminary model based on the specific training sample set of the pair of similar questions, and obtaining the prediction sub-model when the loss function of the prediction preliminary model converges.

9. The method according to claim 1, wherein the random disturbance parameter is generated utilizing the following formula:
$$delta = \frac{1}{1 + exp ( - a )} ;$$
wherein delta represents the random disturbance parameter and a represents a parameter factor, −5≤a≤5.

10. A device for predicting a pair of similar questions comprising:
an input module configured to input a pair of similar questions to be predicted into multiple different prediction models to obtain a prediction result output by each of the prediction models, and add a random disturbance parameter into an embedding layer of at least one of the prediction models; and
an operation module configured to perform voting operation on multiple prediction results to obtain a final prediction result of the pair of similar questions to be predicted;
wherein each of the prediction models comprises a plurality of prediction sub-models, wherein each of the plurality of prediction sub-models is obtained by training the prediction model from a specific training sample set of the pair of similar questions and a training sample set of the pair of similar questions determined by an allocation function;
the input module being further configured to input the pair of similar questions to be predicted into multiple prediction sub-models comprised in each of the prediction models to obtain a prediction sub-result output by each of the prediction sub-models, and performs voting operation on multiple prediction sub-results to obtain the prediction results.

11. The device according to claim 10, wherein the prediction sub-model is trained by the steps of:
obtaining an original training sample set of the pair of similar questions;
performing a training sample extension processing on the original training sample set of the pair of similar questions by utilizing a similarity transmission principle to obtain an extended training sample set of the pair of similar questions;
determining the training sample set of the pair of similar questions from the extended training sample set of the pair of similar questions based on the allocation function;
training the prediction model by utilizing the training sample set of the pair of similar questions and the specific training sample set of the pair of similar questions to obtain the prediction sub-model; and
after the extended training sample set of the pair of similar questions is obtained, sequentially labeling each pair of training samples of the pair of similar questions in the extended training sample set of the pair of similar questions.

12. The device according to claim 11, wherein the step of determining the training sample set of the pair of similar questions from the extended training sample set of the pair of similar questions based on the allocation function comprises:
determining a first label from the extended training sample set of the pair of similar questions by utilizing a first function of the allocation function;
determining a second label from the extended training sample set of the pair of similar questions based on the first label by utilizing a second function of the allocation function; and
selecting an extended training sample set of the pair of similar questions between the first label and the second label as the training sample set of the pair of similar questions.

13. An electronic equipment comprising a processor and a memory, the memory storing computer executable instructions executable by the processor, and the processor executing the computer executable instructions to implement the method according to claim 1.
